(19) United States
(12) Patent Application Publication
(10) Pub. No.: US 2025/0241870 A1
(43) Pub. Date: Jul. 31, 2025

(54) OXYRESVERATROL AND DERIVATIVES THEREOF FOR USE IN LIPOLYSIS

(71) Applicant: Caliway Biopharmaceuticals Co., Ltd., New Taipei City (TW)

(72) Inventor: Yu-Fang LING, New Taipei City (TW)

(21) Appl. No.: 19/036,735

(22) Filed: Jan. 24, 2025

Related U.S. Application Data
(60) Provisional application No. 63/626,657, filed on Jan. 30, 2024.

(51) Int. Cl. A61K 31/05; A61K 31/085; A61K 31/216; A61P 3/04

(52) U.S. Cl. CPC A61K 31/05; A61K 31/085; A61K 31/216; A61P 3/04

(57) ABSTRACT

A method of reducing fat or treating obesity in a subject comprising administering to the subject a pharmaceutical composition comprising an effective amount of a compound of formula (A):

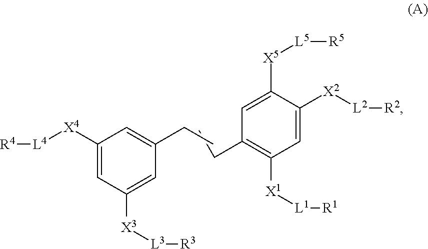

or a pharmaceutically acceptable complex thereof, and a pharmaceutically acceptable carrier; wherein “ [symbol] ”, X¹, X², X³, X⁴, X³, L¹, L², L³, L¹, L⁵, R¹, R², R³, R⁴, and R⁵ are each described herein.

### CROSS REFERENCE TO RELATED APPLICATIONS

This application claims the benefit of the filing date of U.S. Provisional Application No. 63/626,657, filed Jan. 30, 2024, the entire contents of which is incorporated by reference herein.

### BACKGROUND

Overweight and obesity involve abnormal or excessive fat accumulation in the body, raising risks to health. It has been reported that overweight and obesity are associated with various diseases, for example, cardiovascular diseases and diabetes. Rates of overweight and obesity continue to grow in both adults and children, leading to serious health problems worldwide.

The fundamental cause to obesity is an imbalance between calories consumed and calories expended. As such, many existing treatments for obesity aim at suppressing a patient's appetite, thereby reducing calory intake. However, such appetite suppressants can have various side effects and may have limited efficacy.

It is therefore of great importance to develop effective and safe therapies against overweight and obesity.

### BRIEF SUMMARY

The present disclosure is based, at least in part, on the discoveries that exemplary compounds of Formula (A) (e.g., compounds of Formula (I)) provided herein, for example, oxyresveratrol and derivatives thereof, sucessfully inhibited adipocytes at a level greater than that of resveratrol. Accordingly, provided herein are methods for reducing fat in subjects who need the treatment using oxyresveratrol or derivatives thereof as defined herein.

In one aspect, the present disclosure provides a method of reducing fat in a subject in need thereof (e.g., a human patient), the method comprising administering to the subject a pharmaceutical composition comprising an effective amount of a compound of Formula (A):

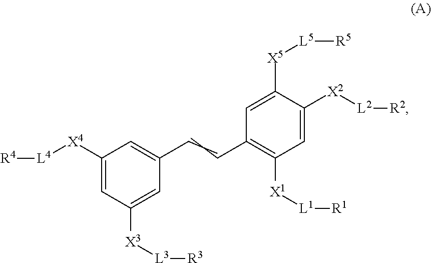

or a pharmaceutically acceptable complex thereof, and a pharmaceutically acceptable carrier. In Formula (A): “ [symbol] ” represents a double bond that has a cis or trans conformation; each of X¹ to X⁵ is independently a bond, O, or S; each of L¹ to L⁵ is independently a bond, C(O), C(O)O, C(O)S, or C(O)NH; and each of R¹ to R⁵ is independently hydrogen, C₁₋₆ alkyl, C₁₋₆ hydroxyalkyl, C₁₋₆ haloalkyl, or C₂₋₆ alkenyl. In some embodiments, when X¹-L¹-R¹ is hydrogen, at least one of X²-L²-R², X³-L³-R³, and X⁴-L⁴-R⁴ is not —OH.

In some embodiments, the Formula (A) compound provided herein may have the structure of Formula (I):

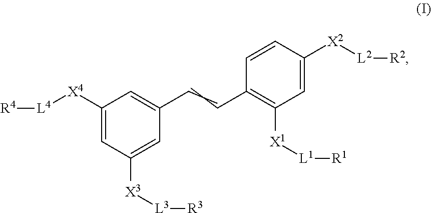

in which “ [symbol] ”, L¹ to L⁴, X¹-X⁴, and R¹-R⁴ are each described above.

In some examples, the compound of Formula (I) is represented by formula (Ia):

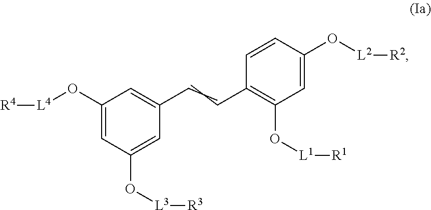

wherein “ [symbol] ”, L¹ to L⁴ and R¹-R⁴ are each described herein.

In specific examples, the compound for use in the method described herein is oxyresveratrol.

In some embodiments, the pharmaceutical composition comprising the Formula (A) compound (e.g., the Formula (I) compounds such as the Formula (Ia) compounds such as oxyresveratrol) may be administered to the subject via a systemic route. In some examples, the pharmaceutical composition can be administered orally. Alternatively, the pharmaceutical composition may be administered to the subject via a local route. For example, the pharmaceutical composition may be administered (e.g., injected) to body site where reducing fact is needed.

In some embodiments, the subject for the treatment may be on a separate therapy involving a fat-reducing agent, which may be different from the compound of Formula (A) (e.g., compounds of Formula (I)) as disclosed herein.

Alternatively, any of the methods disclosed herein may further comprise administering to the subject a second fat-reducing agent, which may be different from the compound of Formula (A) (e.g., compound of Formula (I)) disclosed herein. In some instances, the second fat-reducing agent may be formulated together with the Formula (A) (e.g., Formula (I)) compound in one pharmaceutical composition. Alternatively, the second fat-reducing agent and the Formula (A) (e.g., Formula (I)) compound may be formulated in separate compositions.

In another aspect, the present disclosure provides a method of treating obesity, the method comprising administering to a subject in need thereof a pharmaceutical composition comprising an effective amount of a compound of Formula (A) or formula (I), as described herein.

In some embodiments, the subject may be a human patient having or at risk for obesity. In some embodiments, the human patient may be on a second anti-obesity agent, which may be different from the compound of Formula (A) (e.g., Formula (I)) disclosed herein. Alternatively, the method may further comprise administering to the subject (e.g., the human patient) a second anti-obesity agent, which may be different from the compound of Formula (A) (e.g., Formula (I)) disclosed herein.

Also within the scope of the present disclosure are pharmaceutical compositions comprising a compound of Formula (A) (e.g., Formula (I)) disclosed herein for reducing fat or treatment obesity in a subject in need of the treatment, and use of a compound of formula (A) (e.g., Formula (I)) or a pharmaceutical composition comprising such for manufacturing a medicament for the intended medical uses as provided herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram illustrating the study design for Example 1.

FIGS. 2A-2B include diagrams showing cell viability rates of 3T3-L1 mature adipocytes treated with Resveratrol (Res) or Oxyresveratrol (OxyR) at various concentrations. FIG. 2A: six study groups (treated with Resveratrol or Oxyresveratrol at 20 ppm, 40 ppm, or 60 ppm), as compared to the Control and Vehicle (0.2% DMSO); FIG. 2B: study groups with 40 ppm of Oxyresveratrol relative to the study group with 40 ppm Resveratrol.

FIG. 3 is a diagram showing cell viability rates of 3T3-L1 mature adipocytes treated with Oxyresveratrol (OxyR), Triacetylresveratrol (TriacetylR), and Rhapontigenin (IM05) at 50 ppm as compared to the vehicle (0.2% DMSO).

### DETAILED DESCRIPTION

Adipocytes, also known as lipocytes or fat cells, are the primary type of cells in adipose tissue. Adipocytes form the body's energy storage system by converting metabolites of glucose and fatty acids from blood to triglyceride (TG) for storage via lipogenesis. Lipogenesis is the pathway for fatty acid synthesis from nonlipid precursors and subsequent TG synthesis. Lipolysis, on the other hand, is the process of TG hydrolysis, which results in the release of fatty acid and glycerol into the circulation.

It is reported herein that oxyresveratrol, an exemplary compound of Formula (A) (e.g., Formula (I)) disclosed herein, inhibited adipocytes as shown by decreased cell viability. In particular, at 40 ppm, oxyresveratrol showed better inhibitory effects against adipocytes as compared with resveratrol. Accordingly, the compound of Formula (A) (e.g., Formula (I)) provided herein would be expected to reduce fat in a subject in need of the treatment via, for example, inhibition of adipocytes. Such compounds would also be expected to be effective in treating diseases associated with abnormal or excessive fat accumulation, for example, obesity.

Accordingly, the present disclosure provides methods for reducing fat and/or treating fat-associated diseases such as obesity using one or more of the compound of Formula (A) (e.g., Formula (I)) provided herein, for example, oxyresveratrol.

### Definitions

As used herein, the terms below have the meanings indicated.

Unless specifically indicated otherwise, the group

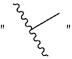

as used herein in any one of formulae of compounds as disclosed herein, refers to methyl.

“Alkyl” refers to a straight or branched, saturated, aliphatic radical having the number of carbon atoms indicated (i.e., C₁₋₆ means one to six carbons). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc.

“Alkenyl” refers to a straight chain or branched hydrocarbon having at least 2 carbon atoms and at least one double bond and having the number of carbon atom indicated (i.e., C₂₋₆ means to two to six carbons). Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₅, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Hydroxyalkyl” refers to an alkyl group, as defined above, where at least one of the hydrogen atoms is replaced with a hydroxy group. As for the alkyl group, a hydroxyalkyl group can have any suitable number of carbon atoms, such as C₁-C₆. Exemplary hydroxyalkyl groups include, but are not limited to, hydroxymethyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3, 4-, 5- or 6-position), 1,2-dihydroxyethyl, and the like.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁-C₆. For example, haloalkyl includes trifluoromethyl, fluoromethyl, 2,2,2-trifluoroethyl, etc. In some instances, the term “perfluoro” can be used to define a compound or radical where all the hydrogens are replaced with fluorine. For example, perfluoromethyl refers to 1,1,1-trifluoromethyl.

“Bond” refers to a covalent linkage between two atoms, or two moieties when the atoms joined by the bond are considered to be part of larger substructure. A bond may be single, double, or triple unless otherwise specified.

“ [symbol] ” represents a double bond that has a cis or trans conformation.

“Pharmaceutically acceptable complex” as used herein refers to a complex including a compound of Formula (A) (e.g., Formula (I)) and at least one complexing agent. The term “pharmaceutically acceptable” means approved by a regulatory agency of the Federal or a state government or listed in the U.S. Pharmacopeia or other generally recognized pharmacopeia for use in animals, and more particularly in humans. The complexing agents have the function to form a complex structure with the compound of Formula (A) (e.g., Formula (I)) through non-covalent secondary interactions. The secondary interactions can form through electrostatic interactions such as ionic interactions, H-bonding, dipole-dipole interactions, dipole-induced dipole interactions, London dispersion forces, p-p interactions, and hydrophobic interactions. The pharmaceutically acceptable complex of the compound of formula (I) includes a pharmaceutically acceptable salt and/or solvate thereof. In some embodiments, a pharmaceutically acceptable complex of a compound of Formula (A) (e.g., Formula (I)) is a pharmaceutically acceptable salt of a compound of Formula (A) (e.g., Formula (I)).

“Salt” refers to acid or base salts of the compounds of the present disclosure. Illustrative examples of pharmaceutically acceptable acid addition salts are mineral acid (hydrochloric acid, hydrobromic acid, phosphoric acid, and the like) salts and organic acid (acetic acid, propionic acid, glutamic acid, citric acid and the like) salts. Examples of pharmaceutically acceptable base addition salts include sodium, potassium, calcium, ammonium, organic amino, or magnesium salt, or a similar salt. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Solvate” refers to a compound that is complexed with a solvent molecule. The compounds of the present invention can be complexed with a stoichiometric or non-stoichiometric amount of solvent bound by non-covalent intermolecular forces. Where the solvent is water, the solvate is a hydrate.

“Hydrate” refers to a compound that is complexed with water molecule. The compounds of the present invention can be complexed with ½ water molecule or from 1 to 10 water molecules.

### I. Compounds of Formula (A)

The oxyresveratrol compounds for use in any of the methods and compositions as disclosed herein can include oxyresveratrol and derivatives thereof, or a pharmaceutically acceptable complex of oxyresveratrol or the derivative thereof. In certain embodiments, the compound for use in the treatment methods provided herein may have the structure of Formula (A):

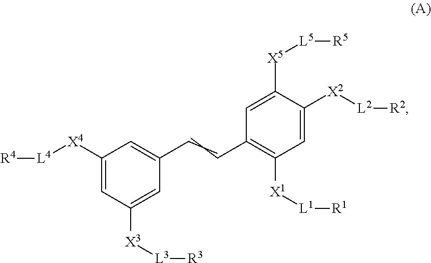

or a pharmaceutically acceptable complex thereof. In Formula (A), “ [symbol] ” represents a double bond that has a cis or trans conformation; each of X¹ to X⁵ is independently a bond, O, or S; each of L¹ to L⁵ is independently a bond, C(O), C(O)O, C(O)S, or C(O)NH; and each of R¹ to R⁵ is independently hydrogen, C₁₋₆ alkyl, C₁₋₆ hydroxyalkyl, C₁₋₆ haloalkyl, or C₂₋₆ alkenyl.

In some instances, when X¹-L¹-R¹ is hydrogen, at least one of X²-L²-R², X³-L³-R³, and X⁴-L⁴-R⁴ is not —OH.

In some instances, at least one of X¹-L¹-R¹, X²-L²_R², X³-L³-R³, X⁴-L⁴-R⁴, and X⁵-L⁵-R⁵ is hydrogen. For example, X⁵-L⁵-R⁵ can be hydrogen. In another example, X¹-L¹-R¹ is hydrogen. In that case, X⁵-L⁵-R⁵ may be —OH and X² may be O, L² may be a bond, and R² may be a C₁₋₆ alkyl group.

In some embodiments, the compound of Formula (A) can be a compound of Formula (I) as shown herein. In certain embodiments, the oxyresveratrol compound is a compound represented by formula (I):

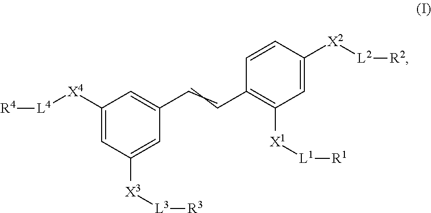

or a pharmaceutically acceptable complex thereof, wherein:
“ [symbol] ” represents a double bond that has a cis or trans conformation;
each of X¹ to X⁴ is independently a bond, O, or S;
each of L¹ to L⁴ is independently a bond, C(O), C(O)O, C(O)S, or C(O)NH; and
each of R¹ to R⁴ is independently hydrogen, C₁₋₆ alkyl, C₁₋₆ hydroxyalkyl, C₁₋₆ haloalkyl, or C₂₋₆ alkenyl,
provided that, when X¹-L¹-R¹ is hydrogen, at least one of X²-L²-R², X³-L³-R³, and X⁴-LA-R⁴ is not —OH.

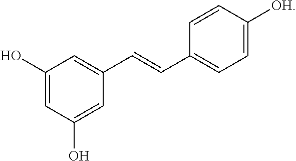 [SMILES: Oc1ccc(/C=C/c2cc(O)cc(O)c2)cc1]

In some embodiments, the compound is not

In some embodiments, “ [symbol] ” represents a double bond that has a cis or trans conformation. In some embodiments, “ [symbol] ” represents a double bond that has a cis conformation. In some embodiments, “ [symbol] ” represents a double bond that has a trans conformation. In some embodiments, each of X¹ to X⁵ is independently a bond, O, or S. In some embodiments, X¹, X², X³, X⁴, and X⁵ are each independently a bond, —O—, or —S—. In some embodiments, each of L¹ to L⁵ is independently a bond, C(O), C(O)O, C(O)S, or C(O)NH. In some embodiments, L¹, L², L³, L⁴, and L⁵ are each independently a bond, —C(O)—, —C(O)O—, —C(O)S—, or —C(O)NH—. In some embodiments, each of R¹ to R⁵ is independently hydrogen, C₁₋₆ alkyl, C₁₋₆ hydroxyalkyl, C₁₋₆ haloalkyl, or C₂₋₆ alkenyl. In some embodiments, R¹, R², R³, R⁴, and R⁵ are each independently hydrogen, C₁₋₆ alkyl, C₁₋₆ hydroxyalkyl, C₁₋₆ haloalkyl, or C₂₋₆ alkenyl. In some embodiments, when X¹-L¹-R¹ is hydrogen, at least one of X²-L²-R², X³-L³-R³, and X⁴-L⁴-R⁴ is not —OH. In some embodiments, when X¹ is a bond, L¹ is a bond, and R¹ is hydrogen, then at least one of the following applies: (i) at least one of X², X³, and X⁴ is not a bond; (ii) at least one of L², L³, and L⁴ is not —O—; and (iii) at least one of R², R³, and R⁴ is not hydrogen.

In some embodiments, one of X¹ to X⁴ is O. In some embodiments, two of X¹ to X⁴ are O. In some embodiments, three of X¹ to X⁴ are O. In some embodiments, each of X¹ to X⁴ is O. In some embodiments, X¹ is O. In some embodiments, one of X¹, X², X³, and X⁴ is —O—. In some embodiments, two of X¹, X², X³, and X⁴ are-O—. In some embodiments, X¹, X², X³, and X⁴ are each —O—.

In some embodiments, X¹-L¹-R¹ is OH. In some embodiments, X¹ is a bond, L¹ is —O—, and R¹ is hydrogen.

In some embodiments, each of R¹ to R⁴ is independently hydrogen or C₁₋₆ alkyl. In some embodiments, R¹, R², R³, and R⁴ are each hydrogen.

In some examples, the compound is represented by formula (Ia):

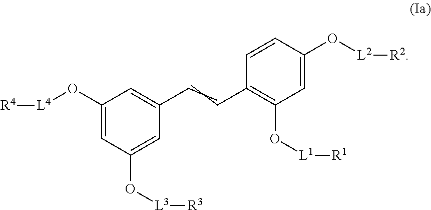

In some embodiments, “ [symbol] ” represents a double bond having a trans conformation, and the compound is represented by the formula:

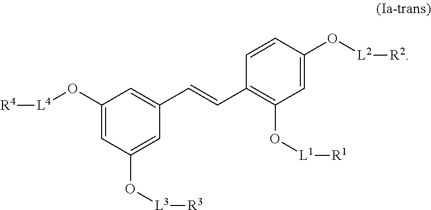

In some embodiments, “ [symbol] ” represents a double bond having a cis conformation, and the compound is represented by the formula:

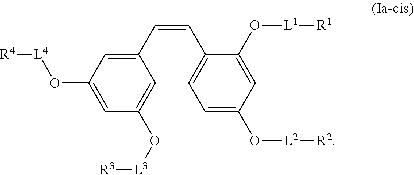

With reference to any one of Formulae (A), (I), (Ia), (Ia-trans), and (Ia-cis), in some embodiments, one of L¹ to L⁴ is a bond. In some embodiments, two of L¹ to L⁴ are each a bond. In some embodiments, three of L¹ to L⁴ are each a bond. In some embodiments, each of L¹ to L⁴ is a bond. In some embodiments, one of L¹, L², L³, and L⁴ is a bond. In some embodiments, two of L¹, L², L³, and L⁴ are each a bond. In some embodiments, three of L¹, L², L³, and L⁴ are each a bond. In some embodiments, each of L¹, L², L³, and L⁴ is a bond.

With reference to any one of formulae (A), (I), (Ia), (Ia-trans), and (Ia-cis), in some embodiments, one of L¹ to L⁴ is C(O). In some embodiments, two of L¹ to L⁴ are each C(O). In some embodiments, three of L¹ to L⁴ are each C(O). In some embodiments, each of L¹ to L⁴ is C(O). In some embodiments, one of L¹, L², L³, and L⁴ is —C(O)—. In some embodiments, two of L¹, L², L³, and L⁴ are each —C(O)—. In some embodiments, three of L¹, L², L³, and L⁴ are each —C(O)—. In some embodiments, each of L¹, L², L³, and L⁴ is —C(O)—.

With reference to any one of formulae (A), (I), (Ia), (Ia-trans), and (Ia-cis), in some embodiments, one of L¹ to L⁴ is C(O)O. In some embodiments, two of L¹ to L⁴ are each C(O)O. In some embodiments, three of L¹ to L⁴ are each C(O)O. In some embodiments, each of L¹ to L⁴ is C(O)O. In some embodiments, one of L¹, L², L³, and L⁴ is —C(O)O—. In some embodiments, two of L¹, L², L³, and L⁴ are each —C(O)O—. In some embodiments, three of L¹, L², L³, and L⁴ are each —C(O)O—. In some embodiments, each of L¹, L², L³, and L⁴ is —C(O)O—.

With reference to any one of formulae (A), (I), (Ia), (Ia-trans), and (Ia-cis), in some embodiments, one of L¹ to L⁴ is C(O)S. In some embodiments, two of L¹ to L⁴ are each C(O)S. In some embodiments, three of L¹ to L⁴ are each C(O)S. In some embodiments, each of L¹ to L⁴ is C(O)S. In some embodiments, one of L¹, L², L³, and L⁴ is —C(O)S—. In some embodiments, two of L¹, L², L³, and L⁴ are each —C(O)S—. In some embodiments, three of L¹, L², L³, and L⁴ are each —C(O)S—. In some embodiments, each of L¹, L², L³, and L⁴ is —C(O)S—.

With reference to any one of formulae (A), (I), (Ia), (Ia-trans), and (Ia-cis), in some embodiments, one of L¹ to L⁴ is C(O)NH. In some embodiments, two of L¹ to L⁴ are each C(O)NH. In some embodiments, three of L¹ to L⁴ are each C(O)NH. In some embodiments, each of L¹ to L⁴ is C(O)NH. In some embodiments, one of L¹, L², L³, and L⁴ is —C(O)NH—. In some embodiments, two of L¹, L², L³, and L⁴ are each —C(O)NH—. In some embodiments, three of L¹, L², L³, and L⁴ are each —C(O)NH—. In some embodiments, each of L¹, L², L³, and L⁴ is —C(O)NH—.

With reference to any one of formulae (A), (I), (Ia), (Ia-trans), and (Ia-cis), in some embodiments, one of L¹ to L⁴ is a bond; and other three of L¹ to L⁴ are each C(O). In some embodiments, two of L¹ to L⁴ are each a bond; and other two of L¹ to L⁴ are each C(O). In some embodiments, one of L¹ to L⁴ is C(O); and other three of L¹ to L⁴ are each a bond. In some embodiments, one of L¹, L², L³, and L⁴ is a bond; and other three of L¹, L², L³, and L⁴ are each —C(O)—. In some embodiments, two of L¹, L², L³, and L⁴ are each a bond; and other two of L¹, L², L³, and L⁴ are each —C(O)—. In some embodiments, one of L¹, L², L³, and L⁴ is C(O); and other three of L¹, L², L³, and L⁴ are each a bond.

With reference to any one of Formulae (A), (Ia), (Ia-trans), and (Ia-cis), in some embodiments, L¹-R¹ is hydrogen; and each of L² to L⁴ is independently a bond, C(O), C(O)O, C(O)S, or C(O)NH; and each of R² to R⁴ is independently hydrogen, C₁₆ alkyl, C₁₋₆ hydroxyalkyl, C₁₋₆ haloalkyl, or C₂₋₆ alkenyl. In some embodiments, L¹-R¹ is hydrogen; and each of L² to L⁴ is independently a bond, C(O), C(O)O, C(O)S, or C(O)NH; and each of R² to R⁴ is independently hydrogen or C₁₋₆ alkyl. In some embodiments, L¹-R¹ is hydrogen; and each of L² to L⁴ is independently a bond, C(O), C(O)O, C(O)S, or C(O)NH; and each of R² to R⁴ is independently hydrogen or C₁₋₃ alkyl. In some embodiments, L¹-R¹ is hydrogen; and each of L² to L⁴ is independently a bond, C(O), C(O)O, C(O)S, or C(O)NH; and each of R² to R⁴ is independently hydrogen, methyl, ethyl, propyl, or isopropyl.

In some embodiments, L¹ is a bond, R¹ is hydrogen, each of L², L³, and L⁴ is independently a bond, —C(O)—, —C(O)O—, —C(O)S—, or —C(O)NH—; and each of R², R³, and R⁴ is independently hydrogen, C₁₋₆ alkyl, C₁₋₆ hydroxyalkyl, C₁₋₆ haloalkyl, or C₂₋₆ alkenyl. In some embodiments, L¹ is a bond, R¹ is hydrogen; each of L², L³, and L⁴ is independently a bond, —C(O)—, —C(O)O—, —C(O)S—, or —C(O)NH—; and each of R², R³, and R⁴ is independently hydrogen or C₁₋₆ alkyl. In some embodiments, L¹ is a bond, R¹ is hydrogen; each of L², L³, and L⁴ is independently a bond, —C(O)—, —C(O)O—, —C(O)S—, or —C(O)NH—; and each of R², R³, and R⁴ is independently hydrogen or C₁₋₃ alkyl. In some embodiments, L¹ is a bond, R¹ is hydrogen; each of L², L³, and L⁴ is independently a bond, —C(O)—, —C(O)O—, —C(O)S—, or —C(O)NH—; and each of R², R³, and R⁴ is independently hydrogen, methyl, ethyl, propyl, or isopropyl.

With reference to any one of Formulae (A), (Ia), (Ia-trans), and (Ia-cis), in some embodiments, L¹-R¹ is hydrogen; and each of L² to L⁴ is independently a bond, C(O), or C(O)O; and each of R² to R⁴ is independently hydrogen or C₁₋₆ alkyl. In some embodiments, L¹-R¹ is hydrogen; and each of L² to L⁴ is independently a bond, C(O), or C(O)O; and each of R² to R⁴ is independently hydrogen or C₁₋₃ alkyl. In some embodiments, L¹-R¹ is hydrogen; and each of L² to L⁴ is independently a bond, C(O), or C(O)O; and each of R² to R⁴ is independently hydrogen, methyl, ethyl, propyl, or isopropyl. In some embodiments, L¹-R¹ is hydrogen; and each of L² to L⁴ is independently a bond, C(O), or C(O)O; and each of R² to R⁴ is independently hydrogen, methyl, or ethyl. In some embodiments, L¹ is a bond, R¹ is hydrogen; each of L², L³, and L⁴ is independently a bond, —C(O)—, or —C(O)O—; and each of R², R³, and R⁴ is independently hydrogen or C₁₋₆ alkyl. In some embodiments, L¹ is a bond, R¹ is hydrogen; each of L², L³, and L⁴ is independently a bond, —C(O)—, or —C(O)O—; and each of R², R³, and R⁴ is independently hydrogen or C₁₋₃ alkyl. In some embodiments, L¹ is a bond, R¹ is hydrogen; each of L², L³, and L⁴ is independently a bond, —C(O)—, or —C(O)O—; and each of R², R³, and R⁴ is independently hydrogen, methyl, ethyl, propyl, or isopropyl. In some embodiments, L¹ is a bond, R¹ is hydrogen; each of L², L³, and L⁴ is independently a bond, —C(O)—, or —C(O)O—; and each of R², R³, and R⁴ is independently hydrogen, methyl, or ethyl.

With reference to any one of Formulae (A), (Ia), (Ia-trans), and (Ia-cis), in some embodiments, L¹-R¹ is hydrogen; and each of L² to L⁴ is independently a bond or C(O); and each of R² to R⁴ is independently hydrogen or C₁₋₆ alkyl. In some embodiments, L¹-R¹ is hydrogen; and each of L² to L⁴ is independently a bond or C(O); and each of R² to R⁴ is independently hydrogen or C₁₋₃ alkyl. In some embodiments, L¹-R¹ is hydrogen; and each of L² to L⁴ is independently a bond or C(O); and each of R² to R⁴ is independently hydrogen, methyl, ethyl, propyl, or isopropyl. In some embodiments, L¹-R¹ is hydrogen; and each of L² to L⁴ is independently a bond or C(O); and each of R² to R⁴ is independently hydrogen, methyl, or ethyl. In some embodiments, L¹ is a bond, R¹ is hydrogen; each of L², L³, and L⁴ is independently a bond or —C(O)—; and each of R², R³, and R⁴ is independently hydrogen or C₁₋₆ alkyl. In some embodiments, L¹ is a bond, R¹ is hydrogen; each of L², L³, and L⁴ is independently a bond or —C(O)—; and each of R², R³, and R⁴ is independently hydrogen or C₁₋₃ alkyl. In some embodiments, L¹ is a bond, R¹ is hydrogen; each of L², L³, and L⁴ is independently a bond or —C(O)—; and each of R², R³, and R⁴ is independently hydrogen, methyl, ethyl, propyl, or isopropyl. In some embodiments, L¹ is a bond, R¹ is hydrogen; each of L², L³, and L⁴ is independently a bond or —C(O)—; and each of R², R³, and R⁴ is independently hydrogen, methyl, or ethyl.

In some specific examples, the compound for use in the methods provided herein can have the structure shown below:

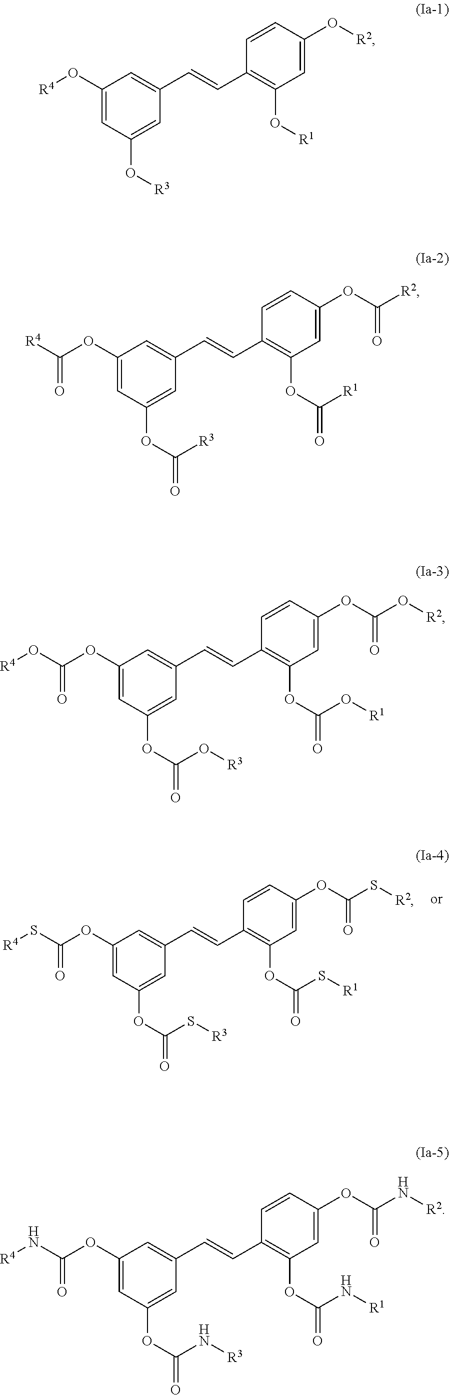

With reference to any one of formulae (Ia-1) to (Ia-5), in some embodiments, each of R¹ to R⁴ is independently hydrogen or C₁₋₆ alkyl. In some embodiments, each of R¹ to R⁴ is independently hydrogen or C₁₋₃ alkyl. In some embodiments, each of R¹ to R⁴ is independently hydrogen, methyl, ethyl, propyl, or isopropyl. In some embodiments, each of R¹ to R⁴ is independently hydrogen, methyl, or ethyl. In some embodiments, each of R¹, R², R³, and R⁴ is independently hydrogen or C₁₋₆ alkyl. In some embodiments, each of R¹, R², R³, and R⁴ is independently hydrogen or C₁₋₃ alkyl. In some embodiments, each of R¹, R², R³, and R⁴ is independently hydrogen, methyl, ethyl, propyl, or isopropyl. In some embodiments, each of R¹, R², R³, and R⁴ is independently hydrogen, methyl, or ethyl.

With reference to formula (Ia-1), in some embodiments, each of R¹ to R⁴ is hydrogen. In some embodiments, one of R¹ to R⁴ is hydrogen; and other three of R¹ to R⁴ are independently C₁₋₆ alkyl. In some embodiments, two of R¹ to R⁴ are hydrogen; and other two of R¹ to R⁴ are independently C₁₆ alkyl. In some embodiments, three of R¹ to R⁴ are hydrogen; and the other one of R¹ to R⁴ is C₁₆ alkyl. In some embodiments, one of R¹ to R⁴ is hydrogen; and other three of R¹ to R⁴ are independently C₁₋₃ alkyl. In some embodiments, two of R¹ to R⁴ are hydrogen; and other two of R¹ to R⁴ are independently C₁₋₃ alkyl. In some embodiments, three of R¹ to R⁴ are hydrogen; and the other one of R¹ to R⁴ is C₁₋₃ alkyl. In some embodiments, R¹ is hydrogen, each of R² to R⁴ is independently hydrogen or C₁₆ alkyl. In some embodiments, R¹ is hydrogen, each of R² to R⁴ is independently hydrogen or C₁₋₃ alkyl. In some embodiments, R¹ is hydrogen; and each of R² to R⁴ is independently hydrogen, methyl, ethyl, propyl, or isopropyl. In some embodiments, R¹ is hydrogen; and each of R² to R⁴ is independently hydrogen, methyl, or ethyl. In some embodiments, each of R¹ to R⁴ is hydrogen. In some embodiments, one of R¹, R², R³, and R⁴ is hydrogen; and other three of R¹, R², R³, and R⁴ are independently C₁₋₆ alkyl. In some embodiments, two of R¹, R², R³, and R⁴ are hydrogen; and other two of R¹, R², R³, and R⁴ are independently C₁₆ alkyl. In some embodiments, three of R¹, R², R³, and R⁴ are hydrogen; and the other one of R¹, R², R³, and R⁴ is C₁₋₆ alkyl. In some embodiments, one of R¹, R², R³, and R⁴ is hydrogen; and other three of R¹, R², R³, and R⁴ are independently C₁₋₃ alkyl. In some embodiments, two of R¹, R², R³, and R⁴ are hydrogen; and other two of R¹, R², R³, and R⁴ are independently C₁₋₃ alkyl. In some embodiments, three of R¹, R², R³, and R⁴ are hydrogen; and the other one of R¹, R², R³, and R⁴ is C₁₋₃ alkyl. In some embodiments, R¹ is hydrogen, each of R², R³, and R⁴ is independently hydrogen or C₁₋₆ alkyl. In some embodiments, R¹ is hydrogen, each of R², R³, and R⁴ is independently hydrogen or C₁₋₃ alkyl. In some embodiments, R¹ is hydrogen; and each of R², R³, and R⁴ is independently hydrogen, methyl, ethyl, propyl, or isopropyl. In some embodiments, R¹ is hydrogen; and each of R², R³, and R⁴ is independently hydrogen, methyl, or ethyl.

With reference to formula (Ia-1), in some embodiments, one of R¹ to R⁴ is hydrogen; and other three of R¹ to R⁴ are methyl. In some embodiments, two of R¹ to R⁴ are hydrogen; and other two of R¹ to R⁴ are methyl. In some embodiments, three of R¹ to R⁴ are hydrogen; and the other one of R¹ to R⁴ is methyl. In some embodiments, R¹ is hydrogen; and each of R² to R⁴ is independently hydrogen or methyl. In some embodiments, one of R¹, R², R³, and R⁴ is hydrogen; and other three of R¹, R², R³, and R⁴ are methyl. In some embodiments, two of R¹, R², R³, and R⁴ are hydrogen; and other two of R¹, R², R³, and R⁴ are methyl. In some embodiments, three of R¹, R², R³, and R⁴ are hydrogen; and the other one of R¹, R², R³, and R⁴ is methyl. In some embodiments, R¹ is hydrogen; and each of R², R³, and R⁴ is independently hydrogen or methyl.

With reference to any one of formulae (Ia-2) to (Ia-5), in some embodiments, each of R¹ to R⁴ is independently C₁₋₆ alkyl. In some embodiments, each of R¹ to R⁴ is independently C₁₋₃ alkyl. In some embodiments, each of R¹ to R⁴ is independently methyl, ethyl, propyl, or isopropyl. In some embodiments, each of R¹ to R⁴ is methyl. In some embodiments, each of R¹ to R⁴ is ethyl. In some embodiments, each of R¹, R², R³, and R⁴ is independently C₁₋₆ alkyl. In some embodiments, each of R¹, R², R³, and R⁴ is independently C₁₋₃ alkyl. In some embodiments, each of R¹, R², R³, and R⁴ is independently methyl, ethyl, propyl, or isopropyl. In some embodiments, each of R¹, R², R³, and R⁴ is methyl. In some embodiments, each of R¹, R², R³, and R⁴ is ethyl.

Exemplified compounds of formula (A) are listed in Table 1:

TABLE 1
Compounds of Formula (A)

| I.D. | Structure |
|---|---|
| 1.001 | 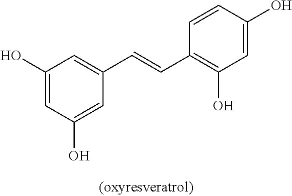 [SMILES: Oc1cc(O)cc(/C=C/c2ccc(O)cc2O)c1] |
| — | / |
| 1.002 | 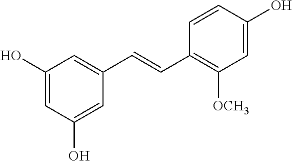 [SMILES: COc1cc(O)ccc1/C=C/c1cc(O)cc(O)c1] |
| 1.003 | 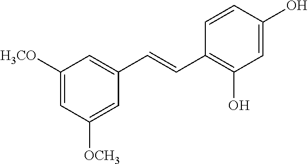 [SMILES: COc1cc(/C=C/c2ccc(O)cc2O)cc(OC)c1] |
| 1.004 | 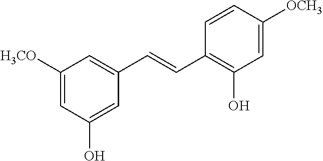 [SMILES: COc1cc(O)cc(/C=C/c2ccc(OC)cc2O)c1] |
| 1.005 | 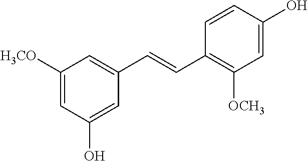 [SMILES: COc1cc(O)cc(/C=C/c2ccc(O)cc2OC)c1] |
| 1.006 | 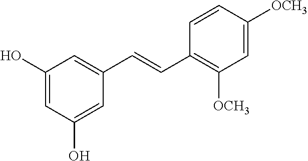 [SMILES: COc1ccc(/C=C/c2cc(O)cc(O)c2)c(OC)c1] |
| 1.007 | 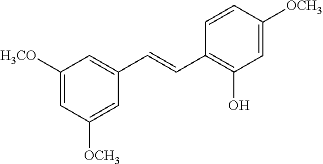 [SMILES: COc1cc(/C=C/c2ccc(OC)cc2O)cc(OC)c1] |
| 1.008 | 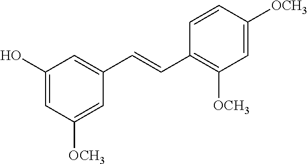 [SMILES: COc1cc(O)cc(/C=C/c2ccc(OC)cc2OC)c1] |
| 1.009 | 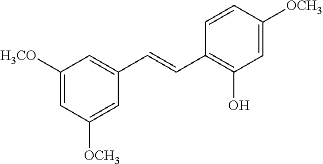 [SMILES: COc1cc(/C=C/c2ccc(OC)cc2O)cc(OC)c1] |
| 2.001 | 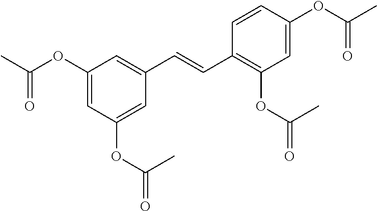 [SMILES: CC(=O)Oc1cc(/C=C/c2ccc(OC(C)=O)cc2OC(C)=O)cc(OC(C)=O)c1] |
| 3.001 | 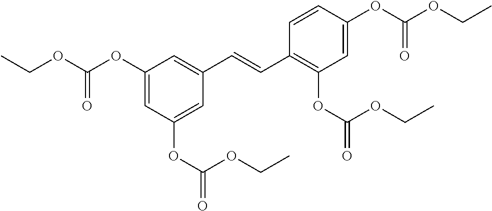 [SMILES: CCOC(=O)Oc1cc(/C=C/c2ccc(OC(=O)OCC)cc2OC(=O)OCC)cc(OC(=O)OCC)c1] |
| 2.002 | 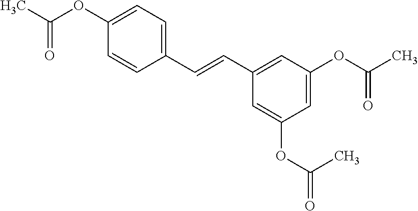 [SMILES: CC(=O)Oc1ccc(/C=C/c2cc(OC(C)=O)cc(OC(C)=O)c2)cc1] |
| 1.010 | 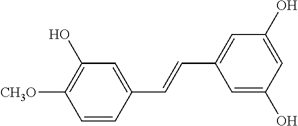 |

The compounds of the present disclosure may exist as complexes with at least one complexing agent. The complexing agents have the function to form a complex structure with the compound of Formula (A) (e.g., Formula (I)) through non-covalent secondary interactions. The secondary interactions can form through electrostatic interactions such as ionic interactions, H-bonding, dipole-dipole interactions, dipole-induced dipole interactions, London dispersion forces, π-π interactions, and hydrophobic interactions. The pharmaceutically acceptable complex of the compound of Formula (A) (e.g., Formula (I)) includes a pharmaceutically acceptable salt, a solvate (including a hydrate), or a combination thereof. In some embodiments, the pharmaceutically acceptable complex of the compound of Formula (A) (e.g., Formula (I)) is a pharmaceutically acceptable salt a solvate, a hydrate, or a combination thereof. In some embodiments, the pharmaceutically acceptable complex of the compound of Formula (A) (e.g., Formula (I)) is a pharmaceutically acceptable salt thereof. In some embodiments, the pharmaceutically acceptable complex of the compound of Formula (A) (e.g., Formula (I)) is a pharmaceutically acceptable solvate thereof. In some embodiments, the pharmaceutically acceptable complex of the compound of Formula (A) (e.g., Formula (I)) is a hydrate thereof. In some embodiments, the pharmaceutically acceptable complex of the compound of Formula (A) (e.g., Formula (I)) is a complex with another pharmaceutically acceptable compound (e.g., polymers, such as polyethylene glycol (PEG), saccharides, etc.).

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Unless otherwise stated, the compounds of the present disclosure may also contain unnatural proportions of atomic isotopes at one or more of the atoms that constitute such compounds. For example, the compounds of the present disclosure may be labeled with radioactive or stable isotopes, such as for example deuterium (²H), tritium (³H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds of the present disclosure, whether radioactive or not, are encompassed within the scope of the present disclosure.

Certain compounds of the present disclosure can readily undergo chemical changes under physiological conditions to provide the parent compounds (e.g., oxyresveratrol). Additionally, certain compounds of the present disclosure can be converted to the parent compounds (e.g., oxyresveratrol) by chemical or biochemical methods in an ex vivo environment. For example, certain compounds of the present disclosure can be slowly converted to the parent compounds (e.g., oxyresveratrol), when administered orally, subcutaneously, transdermally, intradermally, intralesionally, or by implantation with a suitable enzyme or chemical reagent.

### II. Pharmaceutical Compositions

Any of the oxyresveratrol compounds of Formula (A) (e.g., Formula (I)) disclosed here may be mixed with one or more pharmaceutically acceptable carriers to form a pharmaceutical composition, which can be used in the therapeutic applications disclosed herein.

The pharmaceutical compositions as described herein can comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Such carriers, excipients or stabilizers may enhance one or more properties of the active ingredients in the compositions described herein, e.g., bioactivity, stability, bioavailability, and other pharmacokinetics and/or bioactivities.

The carrier in the pharmaceutical composition must be “acceptable” in the sense that it is compatible with the active ingredient of the composition (and preferably, capable of stabilizing the active ingredient) and not deleterious to the subject to be treated. Pharmaceutical excipients useful in the present disclosure include, but are not limited to, binders, fillers, disintegrants, lubricants, coatings, sweeteners, flavors and colors. One of skill in the art will recognize that other pharmaceutical excipients are useful in the present disclosure.

Acceptable carriers (e.g., excipients, or stabilizers) are nontoxic to recipients at the dosages and concentrations used, and may comprise buffers such as phosphate, citrate, and other organic acids; antioxidants including ascorbic acid and methionine; preservatives (such as octadecyl dimethyl benzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; benzoates, sorbate and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, serine, alanine or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ (polysorbate), PLURONICS™ (nonionic surfactants), or polyethylene glycol (PEG).

In some embodiments, the pharmaceutically acceptable carriers comprise a non-ionic surfactant having a hydrophilic-lipophilic balance value (HLB value) greater than 5, a solvent, a buffer agent, an oil phase (e.g., medium-chain triglycerides, or soybean oil), or a combination thereof. In some embodiments, the pharmaceutically acceptable carriers comprise the non-ionic surfactant, which may form nanoparticles with the oxyresveratrol compound of Formula (A) (e.g., Formula (I)). Alternatively, in some embodiments, the pharmaceutical composition comprising the oxyresveratrol compound of Formula (A) (e.g., Formula (I)) contains no nanoparticles. In one embodiment, the pharmaceutical composition has a pH value in the range of 2.5 to 8.0 when dissolved in the water.

In some embodiments, the pharmaceutically acceptable carriers further comprise a co-surfactant, a co-solvent, an antioxidant, or any combination thereof. In some instances, the pharmaceutically acceptable carriers comprise a lipophilic solvent, e.g., triacetin, medium-chain triglycerides, or oil.

In some embodiments, the pharmaceutical composition provided herein is free of water. Alternatively or in addition, in some embodiments, the pharmaceutical composition provided herein is free of antioxidants.

### Non-Ionic Surfactants

The pharmaceutical composition disclosed herein may comprise one or more non-ionic surfactants, which may form nanoparticles with the active agent and/or the hydrophilic therapeutic agent as disclosed herein. Non-ionic surfactants used in the present technology are beneficial at least in the formation of nanoparticles in the composition. In some embodiments, nanoparticles have the effect of encapsulating the active agent and/or the hydrophilic therapeutic agent.

Non-ionic surfactants used in any of the pharmaceutical compositions disclosed herein preferably have a hydrophilic-lipophilic balance (HLB) value greater than 5. The non-ionic surfactant is used in the present technology in a ratio as set forth above. Exemplary non-ionic surfactants include, but are not limited to, polysorbate 80 (Tween® 80), polyoxyl 15 hydroxystearate (Solutol® HS-15), polyoxyethylene castor oil derivatives (e.g., polyoxyl 35 castor oil (Kolliphor® ELP), polyoxyl 40 hydrogenated castor oil (Koliphor® RH40) and polyoxyl 60 hydrogenated castor oil (Cremophor® RH60), Polyoxyethylene (12) glyceryl laurate (UNIGLY ML-212), Polyoxyl 20 Stearate (Myrj™ S20), Polyoxyl 40 Stearate (Myrj™ S40), Polyoxyl 12 Cetostearyl Ether (Kolliphor® CS 12) and Polyoxyl 20 Cetostearyl Ether (Kolliphor® CS 20).

In some instances, the non-ionic surfactant may be a polyoxyethylene derivative, also known as PEGylated excipient. In some examples, the polyoxyethylene derivative is a PEG castor oil derivative, which are materials obtained by reacting varying amounts of ethylene oxide with either castor oil or hydrogenated castor oil. Examples include PEG-35 castor oil and PEG-40 hydrogenated Castor Oil. In other examples, the polyoxyethylene derivative is a PEG ester, which can be manufactured by reacting a polyethylene glycol with a fatty acid. Examples include PEG-40 Stearate and PEG-15 Hydroxystearate. In some instances, the ester may be a sorbitan fatty acid ester (e.g., PEG-20 sorbitan monooleate, PEG-40 sorbitan monooleate, PEG-60 sorbitan monooleate, PEG-80 sorbitan monooleate, PEG-20 sorbitan isostearate, PEG-30 sorbitan tetraoleate, PEG-40, -60 sorbitan tetraoleate, PEG-40 sorbitan diisostearate, or PEG-60 sorbitan tetrastearate), an alkyl glyceride (e.g., PEG-8 Caprylic/capric glycerides, PEG-32 hydrogenated palm glycerides, or PEG-32 Lauroyl glycerides), or an alkyl ether (e.g., PEG-6 cetostearyl ether, PEG-12 cetostearyl ether, PEG-20 cetostearyl ether, PEG-10 cetyl ether, PEG-20 cetyl ether, PEG-4 lauryl ether, PEG-23 lauryl ether, PEG-2 oleyl ether, PEG-10 oleyl ether, PEG-20 oleyl ether, PEG-2 stearyl ether, PEG-10 stearyl ether, PEG-21 stearyl ether, or PEG-100 stearyl ether). In yet other instances, the ester may be a laurate (e.g., PEG-2 laurate, PEG-4 laurate, PEG-6 laurate, PEG-8 laurate. PEG-9-14 laurate, PEG-20 laurate, PEG-32-150 laurate, or PEG-12 glyceryl laurate), a dilaurate (e.g., PEG-2 dilaurate, PEG-4 dilaurate, or PEG-6-150 dilaurate), or a stearate (e.g., PEG-2 stearate, PEG-3 stearate, PEG-4 stearate, PEG-4 isostearate, PEG-5-7 stearate, PEG-6-8 isostearate, PEG-8 stearate, PEG-9 stearate, PEG-10 stearate, PEG-10-isostearate, PEG-12 isostearate, PEG-12-18 stearate, PEG-20 stearate, PEG-23-45 stearate, PEG-40 stearate, PEG-50 stearate, PEG-100 stearate, PEG-75-150 stearate, or PEG-6 and PEG-32 palmitostearate), a glyceryl stearate (e.g., Glyceryl stearate/PEG-40 stearate, Glyceryl stearate/PEG-100 stearate, PEG-120 glyceryl stearate, PEG-20 methyl glucose sesquistearate, or PEG-25 propylene glycol stearate), a distearate (e.g., PEG-2 distearate, PEG-3-120 distearate, PEG-150 distearate, or PEG-175 distearate), a hydroxystearate (e.g., PEG-15 hydroxystearate).

In some instances, the non-ionic surfactant may be a castor oil derivative (e.g., PEG-35 castor oil or PEG-40 castor oil) or a hydrogenated castor oil (e.g., PEG-40 hydrogenated castor oil, PEG-54 hydrogenated castor oil, or PEG-60 hydrogenated castor oil).

Additional examples of non-ionic surfactants include PEG-15 cocamine, Vitamin E polyethylene glycol succinate, PEG-75 lanolin, and PEG-120 methyl glucose dioleate.

### Nanoparticles and Preparations Thereof

In some embodiments, the pharmaceutical compositions disclosed herein comprise nanoparticles formed by one or more non-ionic surfactants as disclosed herein and one or more oxyresveratrol compounds of Formula (A) (e.g., Formula (I)) as also disclosed herein.

In some embodiments, the nanoparticles comprise the non-ionic surfactant and the oxyresveratrol compound at a suitable weight ratio, which lead to suitable particle sizes and/or suitable polydispersity index (PDI) values. For example, a suitable weight ratio between the oxyresveratrol compound and the non-ionic surfactant may range from about 1:1-1:1000, for example, between about 1:1-1:800, between about 1:1-1:600, between about 1:1-1:500, between about 1:1-1:200, or between about 1:1-1:100. In other examples, the suitable weight ratio between the oxyresveratrol compound and the non-ionic surfactant ranges from 1:5 to 1:1000, for example, between 1:5-1:150, between 1:5-1:200, between 1:5-1:300, between 1:5-1:400, between 1:5-1:500, between about 1:8-1:10, between about 1:8-1:20, between about 1:8-1:40, between about 1:8-1:100, between 1:8-1:150, between 1:8-1:200, between 1:8-1:300, between 1:8-1:400, between 1:8-1:500, between about 1:10-1:20, between about 1:10-1:40, between about 1:10-1:100, between 1:10-1:150, between 1:10-1:200, between 1:10-1:300, between 1:10-1:400, between 1:10-1:500, between about 1:20-1:40, between about 1:20-1:100, between 1:20-1:150, between 1:20-1:200, between 1:20-1:300, between 1:20-1:400, between 1:20-1:500, between about 1:40-1:100, between 1:40-1:150, between 1:40-1:200, between 1:40-1:300, between 1:40-1:400, between 1:40-1:500, between 1:100-1:150, between 1:100-1:200, between 1:100-1:300, between 1:100-1:400, between 1:100-1:500, or between 1:150-1:500.

In some instances, the pharmaceutical compositions disclosed herein may comprise nanoparticles having an average size of up to 200 nm, for example, up to 150 nm, up to 100 nm, up to 50 nm, or up to 20 nm. In some examples, the average size of the nanoparticles may range from 5-200 nm, for example, 5-150 nm, 5-100 nm, 5-50 nm, or 5-20 nm.

Alternatively or in addition, the nanoparticles in the pharmaceutical compositions may have a suitable PDI value, for example, up to 0.4. In some examples, the PDI value may range from 0.05-0.4, e.g., 0.05-0.35, 0.05-0.3, 0.05-0.25, 0.05-0.2, 0.05-0.15, or 0.05-0.1.

Any of the nanoparticles disclosed herein can be prepared by conventional methods or as disclosed herein. One example is provided below. A suitable amount of an active agent or a hydrophilic therapeutic agent may be mixed with a suitable solvent (e.g., alcohols (e.g., methanol, ethanol, propanol), acetonitrile, chlorinated solvents (e.g., dichloromethane, chloroform) diethyl ether, and ethyl acetate) and the mixture can be stirred (e.g., at 150-500 rpm) at a suitable temperature until the active agent or the hydrophilic therapeutic agent as disclosed herein is full dissolved in the solvent to form a solution. A suitable amount of a pharmaceutically acceptable non-ionic surfactant (e.g., those disclosed herein) can be added to the solution. The resultant mixture can be stirred (e.g., at 100-300 rpm) under a suitable temperature to volatilize the solvent. Once the solvent is completely volatilized, a suitable amount of a pharmaceutically acceptable aqueous solution (e.g., normal saline) can be added to the mixture to produce micelles having the active agent or hydrophilic agent encapsulated. The resultant particles can be filtered through a suitable filter (e.g., a 0.2 μm filter) and the filtered solution comprising drug-containing micelles can be stored in the dark in a refrigerator for future use.

Additional details on the preparation of the micelles of the present technology can be found in U.S. Pat. No. 10,610,496, the relevant disclosures of which are incorporated by reference herein for the subject matter and purpose referenced herein.

### Additional Components of Pharmaceutical Compositions

In embodiments, the pharmaceutical composition disclosed herein further comprise a co-solvent (e.g., to increase the solubility of drugs), a suspending agent (e.g., to reduce the sedimentation rate of drugs), an oil phase excipient (e.g., to increase the stability of the pharmaceutical composition and the solubility of drugs), an antimicrobial preservative, or a combination thereof.

In some instances, the co-solvent can comprise polyethylene glycol, propylene glycol, ethanol, and other co-solvents, or a combination thereof. Alternatively or in addition, the suspending agent may comprise sodium alginate, glycerol, carboxymethyl cellulose sodium, mannitol, and other suspending agents, or a combination thereof.

In some instances, the oil phase excipient may comprise unsaturated fatty acids, glycerol, triglycerides, and other oil phase excipients, or a combination thereof. For example, the oil phase excipient may comprise one or more unsaturated fatty acids, which may be oleic acid, castor oil, sesame oil, cottonseed oil, soybean oil, safflower oil, corn oil, and other unsaturated fatty acids, or a combination thereof. In some examples, the oil phase excipient may comprise triglycerides, which may be medium chain triglycerides.

In some embodiments, the pharmaceutical composition disclosed herein comprise a local anesthetic. Examples include, but are not limited to, amides, para-aminobenzoic acid esters, and amino ethers, or a combination thereof. In some examples, the local anesthetic comprises amides, which may bedibucaine, lidocaine, mepivacaine HCl, bupivacaine HCl, pyrrocaine HCl, Prilocaine HCl, digammacaine, and oxethazaine, or a combination thereof. In other examples, the local anesthetic comprises para-aminobenzoic acid esters, which may be butacaine, dimethocaine, and tutocaine, or a combination thereof. In yet other examples, the local anesthetic comprises amino ethers, which may be quinisocaine and pramocaine, or a combination thereof.

Alternatively or in addition, the pharmaceutical composition disclosed herein may comprise one or more antioxidant. Examples include, but are not limited to, beta-carotene, lutein, lycopene, bilirubin, vitamin A, vitamin C (ascorbic acid), vitamin E, citric acid, sodium thiosulfate, Propyl gallate, uric acid, nitric oxide, nitroxide, pyruvate, catalase, superoxide dismutase, glutathione peroxidases, N-acetyl cysteine, and naringenin, or a combination thereof. Alternatively, the pharmaceutical composition is free of antioxidants.

Alternatively or in addition, the pharmaceutical compositions provided herein may further comprise a second active agent, which is not an oxyresveratrol compound of Formula (A) (e.g., Formula (I)) disclosed herein. In some instances, the second active agent may be encapsulated in nanoparticles.

In some embodiments, the second active agent comprises a hydrophilic therapeutic agent, for example, green tea extract, epicatechin, epicatechin gallate, epigallocatechin, gallocatechin gallate, gallocatechin, catechin gallate, catechin, epigallocatechin gallate (EGCG), caffeine, carnitine, L-carnitine, synephrine, chlorogenic acid, or a combination thereof.

In some embodiments, the pharmaceutical compositions described herein is formulated as a topical formulation, for example, a cream, lotion, or gel for topical application. Such cream, lotion, or gel may be formulated using ingredients known in the art to be appropriate for topical medications. In some examples, the topical formation may be in the form of a transdermal patch.

In other embodiments, the pharmaceutical compositions described herein is formulated as an injectable formulation (e.g., for intradermal injection). A sterile injectable composition, e.g., a sterile injectable aqueous or oleaginous suspension, can be formulated according to techniques known in the art using suitable dispersing or wetting agents (such as Polysorbate 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In some embodiments, the pharmaceutical composition is in a form of non-oral dosage form, and the subject is a human. For example, the composition formulated for injection can be in the form of powder (e.g., lyophilized powder), sterilized suspension, injectable solution, injectable emulsion, or intravenous fluid. In some examples, the composition maybe placed in a microneedle device for injection. Alternatively, the composition may be formulated for transdermal administration, e.g., in a form of ointment, lotion, liniment, cream, gel, dressing, emulsion, film, patch, poultice, cataplasm, or topical powder.

### Oral Dosage Forms

Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington: The Science and Practice of Pharmacy; Pharmaceutical Press; 22 edition (Sep. 15, 2012).

In certain embodiments, the oral dosage forms are solid and prepared under anhydrous conditions with anhydrous ingredients, as described in detail herein. However, the scope of the compositions provided herein extends beyond anhydrous, solid oral dosage forms. As such, further forms are described herein.

Typical oral dosage forms are prepared by combining the active oxyresveratrol compound of Formula (A) (e.g., Formula (I)) in an intimate admixture with at least one excipient according to conventional pharmaceutical compounding techniques. Excipients can take a wide variety of forms depending on the form of preparation desired for administration. In certain embodiments, excipients suitable for use in oral liquid or aerosol dosage forms include, but are not limited to, water, glycols, oils, alcohols, flavoring agents, preservatives, and coloring agents. In certain embodiments, excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

Because of their case of administration, tablets and capsules represent the most advantageous oral dosage unit forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

In certain embodiments, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

In certain embodiments, excipients that can be used in oral dosage forms include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

In certain embodiments, fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in pharmaceutical compositions is typically present in from about 50 to about 99 weight percent of the pharmaceutical composition or dosage form.

In certain embodiments, suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, PA), and mixtures thereof. A specific binder is a mixture of microcrystalline cellulose and sodium carboxymethyl cellulose sold as AVICEL RC 581. Suitable anhydrous or low moisture excipients or additives include AVICEL PH 103™ and Starch 1500 LM.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, in certain embodiments, a syloid silica gel (AEROSIL 200, manufactured by W.R. Grace Co. of Baltimore, MD), a coagulated aerosol of synthetic silica (marketed by Degussa Co. of Plano, TX), CAB O SIL (a pyrogenic silicon dioxide product sold by Cabot Co. of Boston, MA), and mixtures thereof. If used at all, lubricants are typically used in an amount of less than about 1 weight percent of the pharmaceutical compositions or dosage forms into which they are incorporated.

### Parenteral Dosage Forms

In certain embodiments, provided are parenteral dosage forms. In certain embodiments, parenteral dosage forms can be administered to subjects by various routes including, but not limited to, subcutaneous, intravenous (including bolus injection), intramuscular, and intra-arterial. In certain embodiments, parenteral dosage forms can be administered to subjects by various routes including, but not limited to, topical, intradermal, or intralesional. Because their administration typically bypasses subjects' natural defenses against contaminants, parenteral dosage forms are typically, sterile or capable of being sterilized prior to administration to a subject. In certain embodiments, parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. In certain embodiments, suitable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms.

### Transdermal and Topical Dosage Forms

Also provided are transdermal and topical dosage forms. Transdermal and topical dosage forms include, but are not limited to, sprays, aerosols, creams, lotions, ointments, gels, solutions, emulsions, suspensions, or other forms known to one of skill in the art. Sec, e.g., Remington: The Science and Practice of Pharmacy; Pharmaceutical Press; 22 edition (Sep. 15, 2012); and Introduction to Pharmaceutical Dosage Forms, 4th ed., Lea & Febiger, Philadelphia (1985). Further, transdermal dosage forms include “reservoir type” or “matrix type” patches, which can be applied to the skin and worn for a specific period of time to permit the penetration of a desired amount of active ingredients.

Suitable carriers (e.g., excipients and diluents) and other materials that can be used to provide transdermal and topical dosage forms encompassed herein include the ones known to those skilled in the pharmaceutical arts. Typical carriers include, but are not limited to, water, acetone, ethanol, ethylene glycol, propylene glycol, butane 1,3 diol, isopropyl myristate, isopropyl palmitate, mineral oil, and mixtures thereof to form lotions, tinctures, creams, emulsions, gels or ointments, which are nontoxic and pharmaceutically acceptable. In some embodiments, materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. Moisturizers or humectants can also be added to pharmaceutical compositions and dosage forms if desired. Examples of such additional ingredients are well known in the art. See, e.g., Remington: The Science and Practice of Pharmacy; Pharmaceutical Press; 22 edition (Sep. 15, 2012).

Depending on the specific tissue to be treated, additional components may be used prior to, in conjunction with, or subsequent to treatment with active ingredients provided. In certain embodiments, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates or solvates of the active ingredients can be used to further adjust the properties of the resulting composition.

### III. Methods for Fat Reduction

In one aspect, present disclosure provides a method of reducing fat in a subject in need thereof, the method comprising administering to the subject a pharmaceutical composition comprising an effective amount of a compound of Formula (A) as disclosed herein (e.g., a compound of Formula (I)).

Any of the compounds of Formula (A) (e.g., Formula (I)) such as those provided in Table 1 above (e.g., oxyresveratrol), a pharmaceutically acceptable complex thereof, or a stereoisomer thereof, or a pharmaceutical composition comprising such as disclosed herein can be used to reduce fat in a subject such as a human patient in need of the treatment, for example, to treat, delay the onset, or alleviate the fat in any parts of the body. In some examples, the compound of formula (A) (e.g., Formula (I)) can be oxyresveratrol. In some embodiments, the compound of formula (A) (e.g., Formula (I)) is oxyresveratrol.

In some instances, the subject can be an overweight human patient. Such a human patient may have a body mass weight (BMI) of 25 or above.

In another aspect, the present disclosure provides a method of treating obesity, the method comprising administering to a subject in need thereof a pharmaceutical composition comprising an effective amount of a compound of formula (A) or formula (I), as described herein.

Any of the compound of formula (A) (e.g., Formula (I)), a pharmaceutically acceptable complex thereof, or a pharmaceutical composition comprising such as disclosed herein can be used to treat obesity in a subject such as a human patient in need of the treatment, for example, to treat, delay the onset, or alleviate obesity. In some examples, the compound of formula (A) or formula (I) is oxyresveratrol.

In some instances, the subject can be a human patient having obesity. Such a human patient may have a BMI of 30 or above.

To practice the method disclosed herein, an effective amount of the pharmaceutical composition described herein can be administered to a subject (e.g., a human) in need of the treatment via a suitable route, for example, orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

In some embodiments, the pharmaceutical composition comprising the oxyresveratrol compound of formula (A) (e.g., Formula (I)) is administered to the subject via a systemic route or a local route. In some embodiments, the systemic route includes the administration of the composition orally or parenterally. In some embodiments, the pharmaceutical composition is administered to the subject orally. In some embodiments, the pharmaceutical composition can be in a tablet comprising the oxyresveratrol compound of Formula (A) (e.g., Formula (I)), which can be administered to the subject orally.

In some embodiments, the pharmaceutical composition comprising the oxyresveratrol compound of formula (A) or formula (I) is administered to a body site of the subject where reducing fat is needed. In some embodiments, the pharmaceutical composition is administered to a subject such as a human subject by topical administration or intradermal injection to a body site of the subject where reducing fat is needed. In some instances, the oxyresveratrol or the pharmaceutical composition comprising such may be applied to a skin site where reduction of fat is needed.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of the fat or obesity. Alternatively, sustained continuous release formulations of the oxyresveratrol compound may be appropriate. Various formulations and devices for achieving sustained release are known in the art.

For the purpose of the present disclosure, the appropriate dosage of the pharmaceutical composition as described herein will depend on the specific active agents and optionally hydrophilic agents employed, the type and severity of the disease/disorder, whether the composition is administered for preventive or therapeutic purposes, previous therapy, the patient's clinical history and response to the active agents, and the discretion of the attending physician. Typically, the clinician will administer a pharmaceutical composition disclosed herein, until a dosage is reached that achieves the desired result.

In some embodiments, the desired result is the reduction or elimination fat at any parts of the body or in the body overall. In some embodiments, the desired result is the reduction or elimination obesity in a subject (e.g., human). Methods of determining whether a dosage resulted in the desired result would be evident to one of skill in the art. The particular dosage regimen, i.e., dose, timing and repetition, used in the method described herein will depend on the particular subject and that subject's medical history. Treatment efficacy for reducing fat or treating obesity can be assessed by methods well-known in the art.

In general, an effective treatment (e.g., reducing fat) can be determined by induction of lipolysis or inhibition of adipogenesis in a subject or at a body site of the subject, when treated with the pharmaceutical composition comprising the oxyresveratrol compound of formula (A) or formula (I).

In some embodiments, an effective treatment (e.g., reducing fat) is determined by a weight loss in a subject being treated. In some embodiments, the effective amount of the pharmaceutical composition comprising the oxyresveratrol compound of formula (A) or formula (I) reduces fat in a subject, measured by a weight loss of from about 5% to about 50% as compared to a body weight prior to the treatment. In some embodiments, the effective amount of the pharmaceutical composition comprising the oxyresveratrol compound of formula (A) or formula (I) reduces fat in a subject, measured by a weight loss in about 5%, 10%, 15%, 20%, 25%, 30%, 35%, 40%, 45%, or 50% of a body weight prior to the treatment.

Any of the pharmaceutical compositions disclosed herein may be administered to the subject 1-4 times every day for a suitable period of time, for example, 1 month to 1 year. Duration of the treatment may depend on the treatment results. In some instances, the treatment can be terminated when the fat is reduced to a certain level so that the involved body site or an overall body has a natural body shape. The treatment may resume when fat or obesity occurs again.

Any of the pharmaceutical compositions disclosed herein may be administered with a second agent that is effective for reducing fat and/or treating fat-related diseases such as obesity. In some instances, the second agent is different from the compound of Formula (A) (e.g., Formula (I)). Alternatively, any of the pharmaceutical composition disclosed herein may be administered to a subject who is already on a treatment with the second agent.

The compound of Formula (A) (e.g., Formula (I)) and the second agent can be administered concomitantly or sequentially. In some embodiments, the compound of formula (A) or formula (I) and the second agent are administered concurrently. In some embodiments, the compound of Formula (A) (e.g., Formula (I)) and the second agent are formulated separately in a pharmaceutical composition, both of which are administered concomitantly. In some embodiments, the compound of Formula (A) (e.g., Formula (I)) and the second agent are administered in a pharmaceutical composition comprising the compound and second agent. In some embodiments, the compound of Formula (A) (e.g., Formula (I)) and the second agent are administered sequentially. In some embodiments, the compound of Formula (A) (e.g., Formula (I)) and the second agent are administered sequentially in any order. In some embodiments, the compound of Formula (A) (e.g., Formula (I)) in a pharmaceutical composition is administered prior to the administration of the second agent. In some embodiments, the compound of Formula (A) (e.g., Formula (I)) in a pharmaceutical composition is administered after the administration of the second agent.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that delays or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

### IV. Kits

In one aspect, the present disclosure provides a kit for reducing fat and/or treating a fat-associated disease such as obesity in a subject. The kit may comprise a pharmaceutical composition comprising an effective amount of a compound of Formula (A) (e.g., Formula (I)) as described herein with an instruction for effective administration. Such kits can include one or more containers comprising the pharmaceutical composition disclosed herein. In some embodiments, the pharmaceutical composition is co-used with a second agent that is effective for reducing fat, wherein the second agent is other than the compound of Formula (A) (e.g., Formula (I)).

In some embodiments, the instruction for use is in accordance with any of the methods described herein for reducing fat. The included instructions can comprise a description of administration of the pharmaceutical composition, and optionally the second agent, to treat, delay the onset, or alleviate fat in a subject. The kit may further comprise a description of selecting an individual suitable for the treatment based on identifying whether that individual needs to reduce fat, e.g., following a routine procedure.

The instructions relating to the use of the pharmaceutical composition disclosed herein generally include information as to dosage, dosing schedule, and route of administration for the intended treatment. The containers may be unit doses, bulk packages (e.g., multi-dose packages) or sub-unit doses. Instructions supplied in the kits are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating fat or obesity. Instructions may be provided for practicing any of the methods described herein.

The kits are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. Higgins eds. (1985»; Transcription and Translation (B. D. Hames & S. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Effects of Pharmaceutical Compositions Including Resveratrol or Oxyresveratrol on Lipolysis

### A. Materials

3T3-L1 cell subculture medium (preadipocytes): DMEM+10% BCS+1% P/S.

3T3-L1 cell induction differentiation medium (mature adipocytes): DMI Medium: DMEM+10% FBS+1% P/S+0.5 μM IBMX+0.1 μM dexamethasone+5 μg/mL insulin; and INS Medium: DMEM+10% FBS+1% P/S+5 μg/mL insulin. IBMX stock solution was prepared as follows: 500 mM solution in DMSO diluted and used to prepare the differentiation medium. Dexamethasone stock solution was preprepared as follows: 10 mM solution in EtOH diluted and used to prepare the differentiation medium. Insulin stock solution was prepared as follows: 10 mg/mL solution in double distilled water (ddw) diluted and used to prepare the differentiation medium.

### B. Methods

Steps to induce differentiation: The adipocyte precursors 3T3-L1 cells (purchased from the Food Industry Research and Development Institute, Taiwan; abbreviated as BCRC) were seeded in 12-well plates, such that each well contained 1×10⁵ cells. After two days of culture, the cells were cultured for another two days in a differentiation induction media (DMI medium, wherein contains 0.5 μM of IBMX (purchased from Sigma-Aldrich), 0.1 μM of dexamethasone (purchased from Sigma-Aldrich), and 5 μg/ml of insulin (purchased from Humulin R.). Then, the cells were cultured in an INS medium containing 5 μg/ml of insulin. After that, the INS medium was replaced every two days and differentiation continued until the 7th day. The cell morphology was observed under a microscope. Once the cell morphology changed from spindle-shaped to spherical and many lipid droplets were accumulated in the cells, it indicates that the cells have differentiated into mature adipocytes.

MTT assay: After 3T3-L1 cells were induced to differentiate into mature adipocytes for 8 days, Resveratrol or Oxyresveratrol was added for treatment for 24 hours, then the culture medium was replaced with MTT reagent (working concentration: 0.5 mg/mL), and cultured in a 37° C. incubator for 2 hours. After removing the supernatant, DMSO was added to dissolve the purple crystals, and an ELISA reader was used to measure the OD 570 nm absorbance value and OD 630 nm background absorbance value. The background value OD 630 nm was subtracted from the measured value OD 570 nm in each group to obtain the final absorbance value.

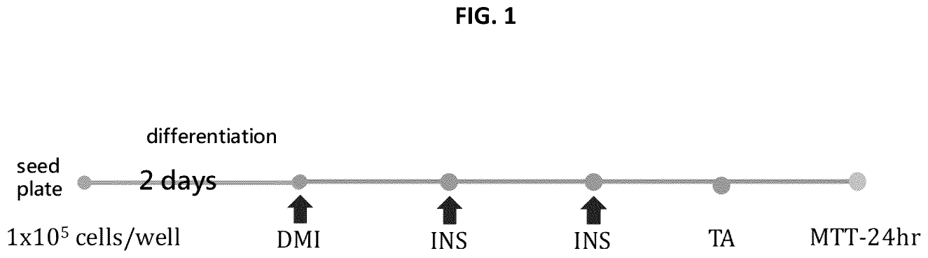

FIG. 1 shows the study design. Table 2 lists the groups in the study.

TABLE 2
Study Groups

| | | | Drug | |
|---|---|---|---|---|
| | | Solvent | concentration | Drug effect |
| No. | Group | concentration | (ppm) | time |
| 1 | Control | — | — | 24 hours |
| 2 | Vehicle | 0.2% DMSO | — | |
| 3 | Resveratrol | 0.2% DMSO | 60 | |
| 4 | Resveratrol | 0.2% DMSO | 40 | |
| 5 | Resveratrol | 0.2% DMSO | 20 | |
| 6 | Oxyresveratrol | 0.2% DMSO | 60 | |
| 7 | Oxyresveratrol | 0.2% DMSO | 40 | |
| 8 | Oxyresveratrol | 0.2% DMSO | 20 | |

Calculation formula (cell viability rate): After measuring the absorbance value at 570 nm and subtracting the background absorbance at 630 nm, the final absorbance value was multiplied by the DMSO re-dissolution volume and divided by the average absorbance value of the Control group for calculation.

### C. Results

The efficacy of Resveratrol or Oxyresveratrol on 3T3-L1 mature adipocytes was measured by a viability rate of 3T3-L1 mature adipocytes—the lower the viability rate of 3T3-L1 mature adipocytes, the better effect of the test substance on reducing fat.

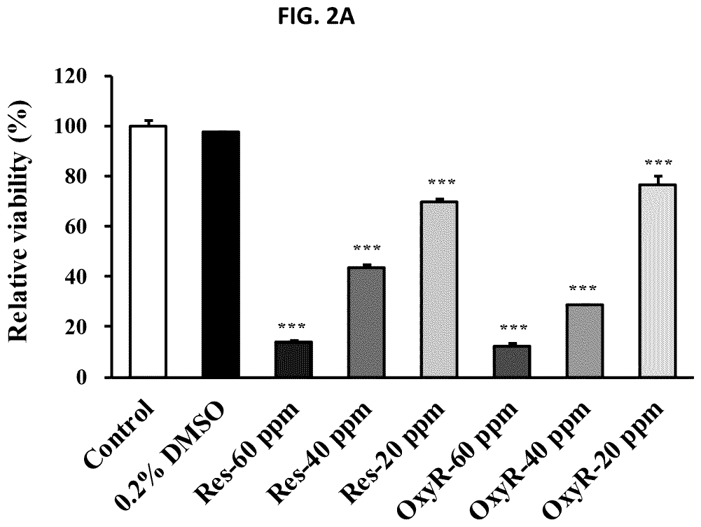

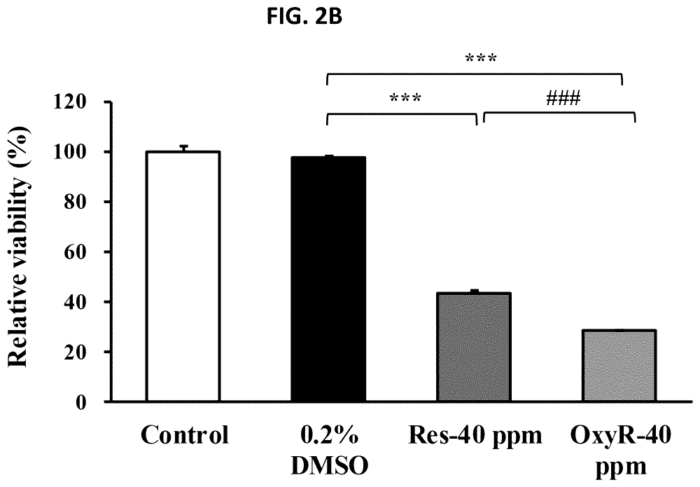

When mature adipocytes were treated with each of Resveratrol (Res) and Oxyresveratrol (OxyR) at concentrations of 20 ppm, 40 ppm and 60 ppm for 24 hours, the cell viability rate decreased with the increase in concentration, showing a dose effect of the treatment, in particular the cell viability rates of treated groups were significantly lower than 0.2% DMSO Vehicle group (p<0.001). The effective treatment of Resveratrol (Res) and Oxyresveratrol (OxyR) were compared at the same concentration. The cell viability rate of the Res-40 ppm group was significantly higher than that of the OxyR-40 ppm group (p<0.001), showing that at a concentration of 40 ppm, OxyR is more effective in reducing adipocytes than Res. At a higher concentration (e.g., 60 ppm), there was no significant difference in cell viability rate between the Res-60 ppm group and the OxyR-60 ppm group. The results are shown in FIGS. 2A to 2B and Table 3.

TABLE 3
Study Results - Cell viability rate of 3T3-L1 mature adipocytes

| | | | | Average |
|---|---|---|---|---|
| Group | Drug Concentration (ppm) | | Replicates | viability (%) |
| Control | — | — | 3 | 100.00 ± 2.36 |
| 0.2% DMSO | — | — | 3 | 97.61 ± 0.26 |
| Res-20 ppm | Resveratrol | 20 ppm | 3 | 69.57 ± 1.36 |
| OxyR-20 ppm | Oxyresveratrol | 20 ppm | 3 | 76.84 ± 3.27 |
| Res-40 ppm | Resveratrol | 40 ppm | 3 | 43.39 ± 1.47 |
| OxyR-40 ppm | Oxyresveratrol | 40 ppm | 3 | 28.65 ± 0.26 |
| Res-60 ppm | Resveratrol | 60 ppm | 3 | 14.07 ± 0.60 |
| OxyR-60 ppm | Oxyresveratrol | 60 ppm | 3 | 12.48 ± 1.09 |

### Example 2: Effects of Pharmaceutical Compositions Including Oxyresveratrol Derivatives on Lipolysis

### A. Materials and Methods

3T3-L1 preadipocytes and mature adipocytes were cultured using the same cell subculture medium disclosed in Example 1 above. Adipocytes differentiation, MTT assay, and determination cell viability was conducted following the methods disclosed herein. See, e.g. Example 1 above.

Table 4 lists the groups in the study.

TABLE 4
Study Groups

| | | | | | Drug | Drug |
|---|---|---|---|---|---|---|
| | | | | Solvent | conc. | effect |
| No. | Group | IUPAC | Structure | conc. | (ppm) | time |
| 1 | Vehicle | — | — | 0.2% | — | 24 hours |
| | | | | DMSO | | |
| 2 | OxyR | 4-[(E)-2-(3,5- dihydroxyphenyl) ethenyl]benzene- 1,3-diol | 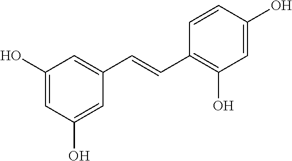 [SMILES: Oc1cc(O)cc(/C=C/c2ccc(O)cc2O)c1] | 0.2% DMSO | 50 | |
| 3 | TriacetylR | [4-[(E)-2-(3,5- diacetyloxyphenyl) ethenyl]phenyl] acetate | 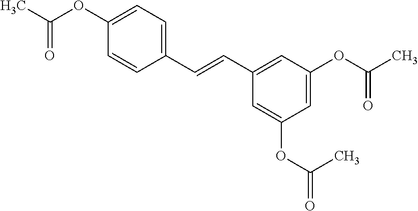 [SMILES: CC(=O)Oc1ccc(/C=C/c2cc(OC(C)=O)cc(OC(C)=O)c2)cc1] | 0.2% DMSO | 50 | |
| 4 | IM05 | 5-[(E)-2-(3-hydroxy- 4-methoxyphenyl) ethenyl]benzene- 1,3-diol | 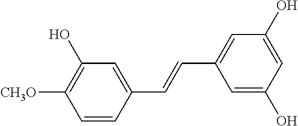 | 0.2% DMSO | 50 | |

### B. Results

As disclosed in Example 1, the efficacy of Oxyresveratrol or its derivatives on 3T3-L1 mature adipocytes was measured by a viability rate of 3T3-L1 mature adipocytes—the lower the viability rate of 3T3-L1 mature adipocytes, the better effect of the test substance on reducing fat.

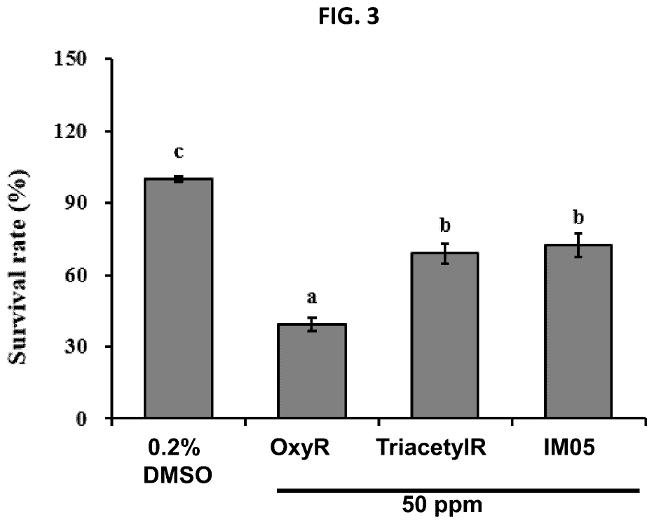

When mature adipocytes were treated with each of Oxyresveratrol (OxyR) or its derivatives Triacetylresveratrol (TriacetylR) or Rhapontigenin (IM05) at concentrations of 50 ppm for 24 hours, the cell viability was significantly decreased compared to the DMSO Vehicle group (p<0.001). Specifically, although the Oxyresveratrol derivatives TriacetylR or IM05 lowered 3T3-L1 survival, Oxyresveratrol was more effective in reducing adipocytes survival than its derivatives (FIG. 3 and Table 5).

TABLE 5
Study Results - Cell viability rate of 3T3-L1 mature adipocytes

Solvent Concentration: 0.2% DMSO

| | | Drug Concentration: 50 ppm | | |
|---|---|---|---|---|
| Group | Vehicle | OxyR | TriacetyIR | IM05 |
| Replicate 1 | 99.9 | 43.2 | 73.2 | 66.5 |
| Replicate 2 | 100.9 | 38.0 | 64.0 | 71.2 |
| Replicate 3 | 98.5 | 37.0 | 67.2 | 73.5 |
| Replicate 4 | 100.7 | 40.0 | 71.2 | 78.4 |
| Mean | 100.0 | 39.6 | 68.9 | 72.4 |
| SD | 1.1 | 2.7 | 4.1 | 5.0 |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method of reducing fat in a subject, comprising administering to a subject in need thereof a pharmaceutical composition comprising an effective amount of a compound of formula (A): 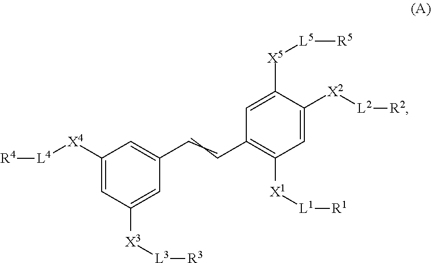
or a pharmaceutically acceptable complex thereof, and a pharmaceutically acceptable carrier;
wherein: [symbol]
represents a double bond that has a cis or trans conformation;
each of X¹ to X⁵ is independently a bond, O, or S;
each of L¹ to L⁵ is independently a bond, C(O), C(O)O, C(O)S, or C(O)NH; and
each of R¹ to R⁵ is independently hydrogen, C₁₋₆ alkyl, C₁₋₆ hydroxyalkyl, C₁₋₆ haloalkyl, or C₂₋₆ alkenyl;
provided that, when X¹-L¹-R¹ is hydrogen, at least one of X²-L²-R², X³-L³-R³, and X⁴-L⁴-R⁴ is not —OH.

2. The method of claim 1, wherein the compound of formula (A) is a compound of formula (I): 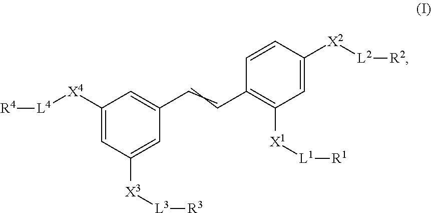
or a pharmaceutically acceptable complex thereof, and a pharmaceutically acceptable carrier;
wherein: [symbol]
represents a double bond that has a cis or trans conformation;
each of X¹ to X⁴ is independently a bond, O, or S;
each of L¹ to L⁴ is independently a bond, C(O), C(O)O, C(O)S, or C(O)NH; and
each of R¹ to R⁴ is independently hydrogen, C₁₋₆ alkyl, C₁₋₆ hydroxyalkyl, C₁₋₆ haloalkyl, or C₂₋₆ alkenyl,
provided that, when X¹-L¹-R¹ is hydrogen, at least one of X²-L²-R², X³-L³-R³, and X⁴-L⁴-R⁴ is not —OH.

3. The method of claim 2, wherein each of X¹ to X⁴ is O; and the compound is represented by formula (Ia):

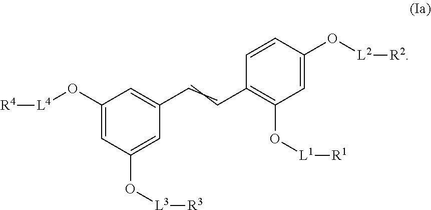

4. The method of claim 2, wherein the compound is represented by the formula selected from the group consisting of:

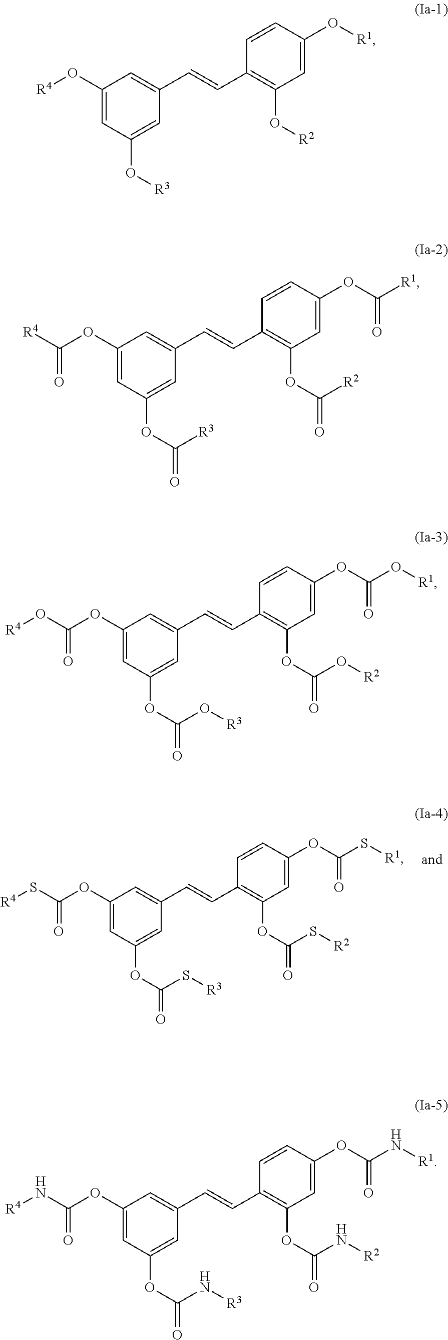

5. The method of claim 2, wherein each of R¹ to R⁴ is independently hydrogen or C₁₋₃ alkyl.

6. The method of claim 2, wherein the compound is represented by the formula:

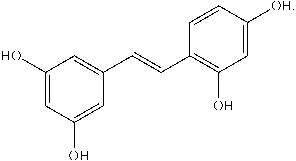 [SMILES: Oc1cc(O)cc(/C=C/c2ccc(O)cc2O)c1]

7. The method of claim 1, wherein the pharmaceutical composition is administered to the subject via a systemic route or a local route.

8. The method of claim 1, wherein the pharmaceutical composition is administered to a body site of the subject where reducing fat is needed.

9. The method of claim 1, wherein the pharmaceutical composition is administered to the subject orally.

10. The method of claim 1, wherein the subject is human.

11. The method of any claim 1, further comprising administering to the subject a second agent that is effective for reducing fat; wherein the second agent is other than the compound of formula (I) in the pharmaceutical composition.

12. The method of claim 11, wherein the pharmaceutical composition comprises the compound of formula (I) and the second agent.

13. A method of treating obesity, comprising administering to a subject in need thereof an effective amount of a pharmaceutical composition comprising an effective amount of a compound according to claim 1 and a pharmaceutically acceptable carrier.

14. The method of claim 13, wherein the subject is a human patient having obesity.

15. The method of claim 13, wherein the subject is on an anti-obesity treatment, which comprises an anti-obesity agent different from the compound of formula (I) in the pharmaceutical composition.

16. The method of claim 13, further comprising administering to the subject an anti-obesity agent, which is different from the compound of formula (I) in the pharmaceutical composition.

17. The method of claim 16, wherein the pharmaceutical composition comprises both the compound of formula (I) and the anti-obesity agent.

18. A pharmaceutical composition for use in reducing fat or treating obesity in a subject, the pharmaceutical composition comprising a compound set forth in claim 1 and a pharmaceutically acceptable carrier.

19. The pharmaceutical composition of claim 18, which further comprises a second therapeutic agent, which is different from the compound; optionally wherein the second therapeutic agent is an anti-obesity agent.
